(19) United States
(12) Patent Application Publication
(10) Pub. No.: US 2025/0025210 A1
(43) Pub. Date: Jan. 23, 2025

(54) MACROENCAPSULATION DEVICE IMPLANTATION AND TRANSPORT SYSTEM AND METHODS

(71) Applicant: Vertex Pharmaceuticals Incorporated, Boston, MA (US)

(72) Inventors: Megan Billings, Warwick, RI (US); John Mills, Warwick, RI (US); John Fraser Mills, Providence, RI (US); Noah Nguyen, Cranston, RI (US); Christopher Thanos, Providence, RI (US)

(73) Assignee: Vertex Pharmaceuticals Incorporated, Boston, MA (US)

(21) Appl. No.: 18/777,686

(22) Filed: Jul. 19, 2024

Related U.S. Application Data
(60) Provisional application No. 63/515,018, filed on Jul. 21, 2023.

(51) Int. Cl. A61B 17/34; A61J 1/03; A61K 9/00

(52) U.S. Cl. CPC A61B 17/3468; A61J 1/03; A61K 9/0024; A61B 2017/347

(57) ABSTRACT

Macroencapsulation implantation devices and containers for storing a macroencapsulation device disposed within a receptacle of a macroencapsulation implantation device as well as related methods are described. In some embodiments, a macroencapsulation implantation device may include a handle that is configured to be selectively engaged with a receptacle including a recess in which a macroencapsulation device may be disposed. In some embodiments, a container may be configured to maintain a receptacle of a macroencapsulation implantation device, and a macroencapsulation device disposed therein, in a desired pose within an internal volume of the container.

### CROSS-REFERENCE TO RELATED APPLICATIONS

This application claims priority under 35 U.S.C. § 119 (c) to U.S. Provisional Application Ser. No. 63/515,018, filed Jul. 21, 2023, and entitled “MACROENCAPSULATION DEVICE IMPLANTATION AND TRANSPORT SYSTEM AND METHODS,” which is incorporated herein by reference in it its entirety for all purposes.

### FIELD

Disclosed embodiments are related to macroencapsulation devices and their associated implantation devices.

### BACKGROUND

Therapeutic devices that deliver biological products can be used to treat metabolic disorders, such as diabetes. These therapeutic devices may be implantable to provide a biological product, such as insulin, for an extended period of time. Some of these devices include macroencapsulation devices which may be used to house cells therein to produce the desired biological product.

### SUMMARY

In some embodiments, a macroencapsulation implantation device may include a receptacle including an internal channel extending at least partially through and along a longitudinal axis of the receptacle. A recess may be formed in a distal portion of the receptacle and the recess may form a distal opening of the internal channel. The recess may be sized and shaped to receive a macroencapsulation device disposed therein. The macroencapsulation implantation device may also include a first portion of a lock formed on a proximal portion of the receptacle. The macroencapsulation implantation device may also include a handle comprising comprise a second portion of the lock configured to be selectively engaged with the first portion of the lock to connect the handle to the receptacle. The macroencapsulation implantation device may also include a pusher configured to be inserted into the internal channel of the receptacle when the handle is connected to the receptacle, and the receptacle may be configured to be displaced proximally relative to the pusher to displace the macroencapsulation device out of the distal opening of the internal channel.

In some embodiments, a method of implanting a macroencapsulation device is provided. The method may include inserting a pusher of a handle into an internal channel of a receptacle and connecting the handle to the receptacle with a lock. The method may also include proximally displacing the receptacle relative to the pusher and handle to displace a macroencapsulation device disposed in a recess formed in a distal portion of the receptacle out of a distal opening of the internal channel.

In some embodiments, a macroencapsulation implantation device may include a receptacle including an internal channel extending at least partially through and along a longitudinal axis of the receptacle. The macroencapsulation implantation device may also include a recess formed in a distal portion of the receptacle and the recess may form a distal opening of the internal channel. The recess may be sized and shaped to receive a macroencapsulation device disposed therein. The macroencapsulation implantation device may also include a plurality of slots formed in the distal portion of the receptacle and extending in a direction substantially parallel to the longitudinal axis of the receptacle. The plurality of slots may be configured to place the recess in fluid communication with a surrounding environment.

In some embodiments, a method of storing a macroencapsulation device is provided. The method may include transferring nutrients and waste between the macroencapsulation device and a surrounding media through a plurality of slots formed in a distal portion of a receptacle that the macroencapsulation device is disposed in.

In some embodiments, a container system for storing a macroencapsulation device is provided. The container system may include a body including an internal volume and an opening as well as a pair of rails positioned on opposing sides of a longitudinal axis of the internal volume and extending into the internal volume. The container system may also include an opposing pair of grooves formed in and extending along at least a portion of a longitudinal length of the pair of rails, and the pair of rails may be configured to retain a correspondingly sized and shaped receptacle engaged with the opposing pair of grooves in a predetermined pose in the internal volume.

In some embodiments, a method of storing a macroencapsulation device is provided. The method may include inserting a receptacle into an opposing pair of grooves formed in and extending along at least a portion of a longitudinal length of a pair of rails positioned on opposing sides of a longitudinal axis of an internal volume of a container. The macroencapsulation device may be disposed in a recessed formed in a distal portion of the receptacle. The method may also include supporting the receptacle in a predetermined pose within the internal volume of the container with the opposing pair of grooves.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A is a top view of a handle of a macroencapsulation implantation device, according to one embodiment;

FIG. 1B is a bottom view of a handle of a macroencapsulation implantation device, according to one embodiment;

FIG. 1C is a perspective view of a handle of a macroencapsulation implantation device, according to one embodiment;

FIG. 2 is a top view of a pusher of a macroencapsulation implantation device, according to one embodiment;

FIG. 3A is a perspective view of a distal portion of a pusher of a macroencapsulation device, according to one embodiment;

FIG. 3B is a second perspective view of the distal portion of the pusher of FIG. 3A, according to one embodiment;

FIG. 4A is a bottom view of an elongated shaft of a macroencapsulation implantation device, according to one embodiment;

FIG. 4B is a perspective view displaying a first side of an elongated shaft of a macroencapsulation implantation device, according to one embodiment;

FIG. 4C is a perspective view displaying a second side of an elongated shaft of a macroencapsulation implantation device, according to one embodiment;

FIG. 4D is a perspective view of a first section of an elongated shaft of a macroencapsulation implantation device, according to one embodiment;

FIG. 4E is a perspective view displaying a second section of an elongated shaft of a macroencapsulation implantation device, according to one embodiment;

FIG. 5 is a top view of a macroencapsulation implantation device, according to one embodiment.

FIG. 6A is a perspective view of a container configured to hold a macroencapsulation implantation device, according to one embodiment;

FIG. 6B is a perspective exploded view of components of a macroencapsulation implantation device within a container, according to one embodiment;

FIG. 6C is a perspective exploded view of a container configured to hold a macroencapsulation implantation device in a predetermined pose, according to one embodiment;

FIG. 6D is a cross section of a front view of a container holding a macroencapsulation implantation device in a predetermined pose, according to one embodiment;

FIG. 7 is a perspective view of a cover, according to one embodiment;

FIG. 8 is a perspective view of an intermediate support configured to engage a macroencapsulation implantation device, according to one embodiment;

FIG. 9A is a perspective view of a cap of a container configured to engage with a proximal end portion of a macroencapsulation implantation device, according to one embodiment;

FIG. 9B is a second perspective view of a cap of a container configured to engage with a proximal end portion of a macroencapsulation implantation device, according to one embodiment;

FIG. 10A is a top view of a macroencapsulation device, according to one embodiment;

FIG. 10B is a cross-sectional side view of an embodiment of a portion of the membranes of a macroencapsulation device in an unfilled configuration; and

FIG. 10C is a cross-sectional side view of an embodiment of a portion of the membranes of a macroencapsulation device in a filled configuration.

### DETAILED DESCRIPTION

Driven by a rising need to deliver biological products to treat various disorders, such as diabetes, different types of implantable therapeutic devices have been engineered. However, such devices, which may include macroencapsulation devices, are often difficult to transport, move, and/or implant within a subject (e.g., a human and/or animal subject). For instance, macroencapsulation devices may include fragile membranes, which may be damaged upon application of sufficiently large forces and/or pressures, and which may cause the overall macroencapsulation device to be damaged and/or contaminated. Due to the therapeutic purposes of the macroencapsulation device, any damage and/or contamination may cause the macroencapsulation device to be unfit for implantation within a subject. Additionally, depending on the type and magnitude of the forces applied to a macroencapsulation device, one or more populations of cells present within the macroencapsulation device may be adversely affected (e.g., through the application of large shear forces which may cause cell injury or death). However, the Inventors have recognized that many traditional methods and systems used for handling an implantable device during transport and implantation typically do not protect the devices from the application of such forces which may damage the macroencapsulation devices and/or potential cell populations disposed therein as described above.

In view of the above, the Inventors have recognized the benefits associated with an implantation device for surgical implantation of macroencapsulation devices which may help to minimize forces and/or pressures applied to a macroencapsulation device while transporting, manipulating, and/or implanting a macroencapsulation device. Specifically, the Inventors have recognized the benefits associated with storing a macroencapsulation device within a recess formed in an elongated channel of a macroencapsulation implantation device prior to implantation. This may include storage and/or transportation of the macroencapsulation device within the elongated shaft. However, due to one or more cell populations being contained within a macroencapsulation device in some embodiments, it may be desirable to store the elongated shaft and macroencapsulation device disposed therein in appropriate media to provide nutrients to the one or more cell populations and remove waste from the one or more cell populations. This transport of nutrients and waste may occur between the media surrounding the elongated shaft of the macroencapsulation implantation device and the macroencapsulation device through one or more membranes of the macroencapsulation device.

While it may be desirable to house a macroencapsulation device within a recess of an elongated shaft of a macroencapsulation implantation device prior to implantation, it may not be practical to store the macroencapsulation device with an entire macroencapsulation implantation device while also immersing the macroencapsulation device within a desired media. Accordingly, in some embodiments, a macroencapsulation implantation device may include an elongated shaft that is configured to be selectively removed from and attached to an associated handle. When removed from the handle, the elongated shaft, which may include a recess sized and shaped to receive a macroencapsulation device therein, may be sized and shaped to be disposed within an interior volume of a container including an appropriate media disposed therein. The container may be configured to support the elongated shaft in a desired pose within the interior volume of the container to help avoid occluding exposed portions of the macroencapsulation device and/or applying undesirable forces to the macroencapsulation device while stored within the elongated shaft in the container. For example, various types of supports may be used including, but not limited to rails, detents, spacers, covers, grips, or other types of supports that may be configured to engage with the elongated shaft to maintain the elongated shaft and macroencapsulation device disposed therein in the desired pose. In some embodiments, the desired pose may correspond to a pose of the elongated shaft that is at least partially positioned within the interior volume of the container and oriented in a direction that is substantially parallel to a longitudinal axis of the interior volume of the container.

To facilitate the above noted storage of an elongated shaft of a macroencapsulation implantation device within the interior volume of a container, a macroencapsulation implantation device may include both a handle and an elongated shaft. The handle may include a pusher, which may extend in a distal direction away from a portion of the handle configured to be gripped or otherwise engaged with by a user and/or robotic surgical system. The pusher may be configured to be inserted into an internal channel extending along a length of the elongated shaft. When the pusher is disposed within the internal channel of the elongated shaft, the handle may be selectively attached to the elongated shaft using a lock. A first portion of the lock may be formed on a proximal portion of the elongated shaft and a second portion of the lock may be formed on the handle. As such, the elongated shaft and the handle may be selectively attached to and/or removed from each other using the lock. Therefore, the entire elongated shaft may be attached to or removed from the handle, which may permit the storage of the elongated shaft within the above noted container including an internal volume at least partially filled with a desired media.

In some embodiments, it may be desirable to use the lock to selectively permit or prevent relative movement of the elongated shaft and handle of a macroencapsulation implantation device. For example, as elaborated on further below with regards to the figures, a lock may be moved between a first unlocked configuration and a second locked configuration when the pusher of the handle is inserted into the internal channel of the elongated shaft. This may selectively permit the elongated shaft to be retained on or removed from the handle. Additionally, in some embodiments, when attached to the handle and the lock is in the second locked configuration, the elongated shaft may be configured to be displaced in a proximal direction relative to the pusher of the handle. However, when the lock is in the first unlocked configuration, proximal movement of the elongated shaft relative to the pusher may be prevented beyond the initial insertion of the pusher into the internal channel of the elongated shaft. A safety also configured to prevent movement of the elongated shaft relative to the handle may also be used in some embodiments. Both of these features may help to prevent unintentional and/or partial deployments of a macroencapsulation device during operation.

As noted above, a macroencapsulation device may be exposed to a desired media during storage within a container containing the media. However, diffusion of the media with the macroencapsulation device may be impeded in instances in which a non-porous elongated shaft is used. Accordingly, in some embodiments, the Inventors have recognized the advantages associated with the use of one or more slots formed in a portion of the elongated shaft in which the recess configured to contain the macroencapsulation device is formed. The one or more slots formed in the elongated shaft may place the recess, and thus the macroencapsulation device disposed therein, in fluid communication with a surrounding environment of the elongated shaft. In some embodiments, the one or more slots are a plurality of slots including one or more slots formed on opposing sides of the elongated shaft. For example, a first group of slots may be formed on a first surface of a distal portion of the elongated shaft including the recess and a second group of slots may be formed on a second surface of the elongated shaft opposite the first surface. In either case, the one or more slots may be sized and shaped to permit sufficient diffusion of nutrients and waste between an interior volume of the macroencapsulation device and the surrounding environment through the one or more slots to maintain the viability of one or more cell populations contained within the macroencapsulation device. In some instances, the plurality of slots may be elongated slots that extend in a direction parallel to a longitudinal axis of the elongated shaft which may help avoid abrasion of a subject's tissue during insertion of the elongated shaft into a target surgical site. In some embodiments, each slot of the plurality of slots may be a linear slot, though other suitable shapes including but not limited to ovals, circles, squares, and/or other non-linear shapes may also be used as the disclosure is not so limited.

In the various embodiments disclosed herein, the plurality of slots may have a combined area (in a plane parallel to a surface of the macroencapsulation device) that is greater than or equal to comprise between and or equal to 30%, 40%, 50%, 60%, or other appropriate percentage of a corresponding total surface area of the macroencapsulation device and/or recess. Accordingly, in some embodiments, the plurality of slots may have a combined area that is greater than or equal to 30% of a corresponding total surface area of the macroencapsulation device and/or recess. In some embodiments, the plurality of slots may have a combined area that is greater than or equal to 40% of a corresponding total surface area of the macroencapsulation device and/or recess. In some embodiments, the plurality of slots may have a combined area that is greater than or equal to 50% of a corresponding total surface area of the macroencapsulation device and/or recess. In some embodiments, the plurality of slots may have a combined area that is greater than or equal to 60% of a corresponding total surface area of the macroencapsulation device and/or recess. The combined area of the plurality of slots may also be less than or equal to 80%, 70%, 60%, 50%, 40%, or other appropriate percentage of the corresponding total surface area of the macroencapsulation device and/or recess. Accordingly, in some embodiments, the combined area of the plurality of slots may be less than or equal to 80% of the corresponding total surface area of the macroencapsulation device. In some embodiments, the combined area of the plurality of slots may be less than or equal to 70% of the corresponding total surface area of the macroencapsulation device. In some embodiments, the combined area of the plurality of slots may be less than or equal to 60% of the corresponding total surface area of the macroencapsulation device. In some embodiments, the combined area of the plurality of slots may be less than or equal to 50% of the corresponding total surface area of the macroencapsulation device. In some embodiments, the combined area of the plurality of slots may be less than or equal to 40% of the corresponding total surface area of the macroencapsulation device. Combinations of the above ranges are contemplated including, for example, a combined area of the plurality of slots that is between or equal to 30% and 80% of the corresponding total surface area of the macroencapsulation device and/or recess.

Although the term “elongated shaft” is frequently used herein to denote a portion of an implantation device that facilitates storage, transportation, and/or deployment of a macroencapsulation device, the macroencapsulation device may alternatively be stored in, transported in, and/or deployed from another type of receptacle that is configured to selectively engage with a handle of an implantation device as described herein (e.g., using a lock or other arrangement). A receptacle may include an internal channel configured to receive a pusher of the handle as described herein, a recess sized and shaped to receive a macroencapsulation device as described herein, and/or a distal opening formed by the recess as described herein. A receptacle may be formed in any appropriate geometry, including an elongated shaft as described herein and/or any other appropriate geometry.

The use of an elongated shaft or other receptacle for both delivering and storing a macroencapsulation device may offer multiple benefits. For example, reduced handling of the macroencapsulation device may help minimize a potential for contamination and/or damage of the macroencapsulation device both prior to and during implantation. For example, storage and delivery from within a recess of the elongated shaft/receptacle may obviate the need for a medical practitioner to manipulate the macroencapsulation device prior to implantation while also helping to shield the macroencapsulation device from the inadvertent application of potentially damaging forces and/or contacts. Of course, it should be understood that the current disclosure is not limited to just these benefits and other benefits different from those noted above are also possible.

Appropriate materials for use with any one of the embodiments of a macroencapsulation implantation device and/or associated container disclosed herein may include but are not limited to a biocompatible plastics, metals, ceramics, and/or combinations of the forgoing capable of being used for the applications disclosed herein. For example, suitable materials may include, but are not limited to, aluminum, titanium, stainless steel, alumina, silicone, polycarbonate, polyvinylchloride (PVC), polypropylene (PP), polyether ether ketone (PEEK), polyurethane (PU), and polyethylene (PE). In some embodiments, components of the macroencapsulation implantation device and associated container may also be treated and/or coated to modify properties of the material, such as chemical resistance and/or color. For example, components of the macroencapsulation implantation device and/or associated container constructed at least in part from aluminum may include an anodized coating. It should be understood that the various components of macroencapsulation implantation devices and the associated containers may be made from any appropriate combination of materials as the disclosure is not limited to being made from any specific material.

In some embodiments, a macroencapsulation implantation device and/or an associated container is sterile. The macroencapsulation implantation device and/or associated container may be configured as a single-use sterile device or as a reusable sterilizable device. A reusable device may be used multiple times on the same subject and/or it may be sterilized in between each use, especially in between different uses for different subjects. In some embodiments, only a portion of the macroencapsulation implantation device, e.g., the handle, may be reusable and sterilizable, while other components, e.g., the elongated shaft and pusher, may be single use. Additionally, some components of the macroencapsulation implantation device, such as the handle, may not directly contact a biological material in some embodiments, and may be removably attachable to a pusher and/or elongated shaft, which may directly contact a biological material. The Inventors have recognized that such a releasable coupling between these components may permit the use of separate sterile components with a single handle in some applications.

For any reusable component which is sterilizable, the component may be made from any appropriate material for a desired type of sterilization. Possible sterilization methods may include but are not limited to heat sterilization, chemical sterilization, and/or radiation sterilization which include methods such as moist heat (autoclave), dry heat, flash steam, performic acid, peracetic acid, formaldehyde, carbon dioxide, ethylene oxide, ozone, plasma, and ultraviolet light.

As used herein, a user of a macroencapsulation implantation device may refer to an individual who may store, transfer, implant, and/or otherwise manipulate the macroencapsulation device either prior to or during a surgical procedure. In some embodiments, a user may refer to a surgeon and/or medical practitioner, and the macroencapsulation implantation device may be used during surgery and/or other medical procedures.

A macroencapsulation device may include multiple layers of membranes. At least one exterior membrane of these multiple layers of membranes may be semipermeable. However, embodiments in which each of the membranes is semipermeable or where at least one of the membranes within a device are substantially impermeable are also contemplated. Further, a device may include two stacked membranes, three stacked membranes, and/or any other appropriate number of membranes as the disclosure is not limited in this fashion. For example, in one embodiment including two membranes, either membrane may be semipermeable and the other impermeable or both may be semipermeable. Accordingly, it should be understood that the current disclosure is not limited to any particular combination of membranes within a stacked structure. Exemplary macroencapsulation devices include, for example, those described in WO2018232180, WO2019068059, WO2020206150, WO2020206157, and WO2023023006, each of which is incorporated-by-reference in its entirety.

In some embodiments, a macroencapsulation device may include at least one population of cells disposed within an internal volume of the device. For example, the population of cells may be disposed within an internal volume formed between two or more opposing layers of one or more exterior membranes of the device where an exterior edge of the internal volume may be defined by one or more bonds extended around at least a portion, and in some instances an entire, perimeter of the membranes or other appropriate portion of the membranes. In such an embodiment, at least the exterior membranes of the device may be configured to block passage of the one or more populations of cells out of the device. Accordingly, the one or more populations of cells may be retained within the interior volume of the device. While the use of two exterior membranes forming a single internal volume is primarily described, the use of multiple intermediate membranes positioned between the exterior membranes of a device and/or multiple unconnected interior volumes within a device are also contemplated. Additionally, instances in which a single membrane is folded over and bonded to itself to provide two opposing membranes to form the interior volume are also contemplated.

Although expanded polytetrafluoroethylene (ePTFE) may be used as a membrane material, the membranes of a macroencapsulation device may be formed from any appropriate biocompatible material. The biocompatible material may be substantially inert towards cells housed within the macroencapsulation device and the surrounding tissue. The biocompatible material may comprise a synthetic polymer or a naturally occurring polymer. In some embodiments, the polymer may also be a linear polymer, a cross linked polymer, a network polymer, an addition polymer, a condensation polymer, an elastomer, a fibrous polymer, a thermoplastic polymer, a non-degradable polymer, combinations of the foregoing, and/or any other appropriate type of polymer as the disclosure is not limited in this fashion. As noted above, in one embodiment, a polymer may comprise expanded polytetrafluoroethylene (ePTFE). Appropriate types of polymers may also comprise polyvinylchloride (PVC), polyethylene (PE), polypropylene (PP), polymethylmethacrylate (PMMA), polystyrene (PS), polytetrafluoroethylene (PTFE), expanded polytetrafluoroethylene (ePTFE), polyurethane (PU), polyamide (nylon), polyethyleneterephthalate (PET), polyethersulfone (PES), polyetherimide (PEI), polyvinylidene difluoride (PVDF), polycaprolactone (PCL), poly (lactic-co-glycolic acid) (PLGA), poly-L-lactide (PLLA), polyacrylonitrile (PAN), electrospun PAN/PVC, any combination of the foregoing, and/or any other appropriate polymeric material. In some embodiments, a membrane used with any of the embodiments disclosed herein may comprise PVDF. In some embodiments, a membrane used with any of the embodiments disclosed herein may comprise electrospun PAN PVC. In some embodiments, a membrane used with any of the embodiments disclosed herein may comprise PES. In some embodiments, a membrane used with any of the embodiments disclosed herein may comprise PS. In some embodiments, a membrane used with any of the embodiments disclosed herein may comprise PAN. In some embodiments, a membrane used with any of the embodiments disclosed herein may comprise Polycarbonate. In some embodiments, a membrane used with any of the embodiments disclosed herein may comprise polypropylene. In some embodiments, a membrane used with any one of the embodiments disclosed herein may comprise PVC. In some embodiments, a membrane used with any one of the embodiments disclosed herein may comprise PU. In some embodiments, a membrane used with any one of the embodiments disclosed herein may comprise PET. In some embodiments, a membrane used with any one of the embodiments disclosed herein may comprise PCL. In some embodiments, a membrane used with any one of the embodiments disclosed herein may comprise PLGA. In some embodiments, a membrane used with any one of the embodiments disclosed herein may comprise PLLA. In some embodiments, a membrane used with any one of the embodiments disclosed herein may comprise PMMA. In some embodiments, a membrane used with any one of the embodiments disclosed herein may comprise PEI. In some embodiments, a membrane used with any one of the embodiments disclosed herein may comprise nylon. In some embodiments, a membrane used with any one of the embodiments disclosed herein may comprise PTFE. In some embodiments, a membrane used with any one of the embodiments disclosed herein may comprise PE. The synthesis methods used for forming one or more of the porous membranes from the above noted polymeric materials may include, but are not limited to, expansion, solvent-casting, immersion precipitation and phase separation, electrospinning, methods that yield isoreticular networks, methods that yield trabecular networks, or any other appropriate method of forming a porous polymer membrane.

Sintering of a membrane may be used to alter the porosity and flux properties of a membrane. For example, the sintering may increase the porosity of the membrane while maintaining its pore structure. The sintering may also improve the mechanical stability and diffusive flux of the membrane. In some instances, a sintered membrane can have a lower melting temperature than an unsintered membrane of the same type. Further, sintered membranes may exhibit a different energy release during a differential scanning calorimetry scan, indicating a more relaxed structure in addition to the thickened porous network exhibited in sintered materials.

In view of the above, sintering may be used to alter the porosity and/or mechanical properties of the membranes, which in turn can be used to tune the porosity and the flux properties of the macroencapsulation device. Accordingly, in some embodiments, any desired combination of sintered and/or unsintered membranes or membrane layers may be used. For instance, two exterior membrane layers of a device may be bonded together where either a sintered and unsintered membrane are bonded together, two sintered membranes are bonded together, or two unsintered membranes are bonded together. Further, any number of intermediate membranes positioned between these exterior membranes may be used where these intermediate membranes may be sintered or unsintered.

The membranes of a macroencapsulation device as described herein may be made from porous membrane materials that are configured to allow for transport through the membranes of materials, such as a biological product, with a molecular weight less than about 3000 kDa, 2000 kDa, 1000 kDa, 500 kDa, 400 kDa, 300 kDa, 200 kDa, 100 kDa, 50 kDa, 40kDa, 30 kDa, 20 kDa, 10 kDa, 6 kDa, 5 kDa, 4 kDa, 3 kDa, 2 kDa, 1 kDa, and/or any other appropriate range of molecular weights depending on the desired application. The membranes of a macroencapsulation device as described herein may be made from porous membrane materials that are configured to allow for transport through the membranes of materials, such as a biological product, within the molecular weight range of 1-3000 kDa, 1-2000 kDa, 1-1000 kDa, 1-500 kDa, 1-400 kDa, 1-300 kDa, 1-200 kDa, 1-100 kDa, 1-50 kDa, 1-40 kDa, 1-30 kDa, 1-20kDa, 1-10 kDa, 1-6 kDa, 1-5 kDa, 1-4 kDa, 1-3 kDa, or 1-2 kDa. For example, the one or more membranes of a macroencapsulation device may be configured to permit the flow of insulin through the membranes which has a molecular weight of about 5.8 kDa. In some embodiments, the one or more membranes of a macroencapsulation device may be configured to permit the flow of materials, such as a biological product, within the range of 1-10 kDa. In some embodiments, the one or more membranes of a macroencapsulation device may be configured to permit the flow of materials, such as a biological product, within the range of 1-6 kDa. In some embodiments, the one or more membranes of a macroencapsulation device may be configured to permit the flow of materials, such as a biological product, within the range of 1-5 kDa. In some embodiments, the one or more membranes of a macroencapsulation device may be configured to permit the flow of materials, such as a biological product, within the range of 1-4 kDa. In some embodiments, the one or more membranes of a macroencapsulation device may be configured to permit the flow of materials, such as a biological product, within the range of 1-3 kDa. In some embodiments, the one or more membranes of a macroencapsulation device may be configured to permit the flow of materials, such as a biological product, within the range of 1-2 kDa.

To provide the desired selectivity, the porous membranes used with the macroencapsulation devices disclosed herein may have an open porous structure (i.e., a structure including a plurality of interconnected pores) with average pore sizes that are greater than or equal to about 1 nm, 5 nm, 10 nm, 15 nm, 20 nm, 30 nm, 40 nm, 50 nm, 60nm, 70 nm, 80 nm, 90nm, 100 nm, 200 nm, 300 nm, and/or any other appropriate size range. Correspondingly, the average pore size of the various membranes described herein may have an average pore size that is less than or equal to 2500 nm, 2000 nm, 1700 nm, 1500 nm, 1400 nm, 1300 nm, 1200 nm, 1100 nm, 1000 nm, 900 nm, 800 nm, 700 nm, 600 nm, 500 nm, 400 nm, 300 nm, 200 nm, 100nm, 90 nm, 80 nm, 70 nm, 60 nm, 50 nm, 40 nm, 30 nm, 20 nm, and/or any other appropriate size range. Combinations of the foregoing are contemplated including, for example, an average pore size that is between or equal to 1 nm and 20 nm, 1 nm and 2500 nm, 50 nm and 1200 nm, and/or any other appropriate combination. In some embodiments, the average pore size of the various membranes described herein is between 25 nm and 1500 nm. In some embodiments, the average pore size of the various membranes described herein is between 50 nm and 1200 nm. In some embodiments, the average pore size of the various membranes described herein is between 50 nm and 1000 nm. In some embodiments, the average pore size has an upper size limit of 1500 nm. In some embodiments, the average pore size has an upper size limit of 1200 nm. In some embodiments, the average pore size has a lower size limit of 25 nm. In some embodiments, the average pore size has a lower size limit of about 50 nm. While specific average pore sizes are described above, it should be understood that any appropriate average pore size may be used for the various membranes described herein including average pore sizes both greater than and less than those noted above.

In some embodiments, a cell population contained within a compartment of a macroencapsulation device may be an insulin secreting cell population. In some embodiments, a cell population contained within a compartment of a macroencapsulation device comprises a heterogeneous population of cells. In some embodiments, the cell population comprises at least one cell derived from a stem cell derived cell. In some embodiments, at least one cell is a genetically modified cell. In some cases, at least one cell is genetically engineered to reduce an immune response in a subject upon implantation of the device, as compared to comparable cells that are not genetically engineered. In some embodiments, the cell population is a stem cell derived cell that is capable of glucose-stimulated insulin secretion (GSIS). For example, an appropriate population of cells may comprise pancreatic progenitor cells, endocrine cells, beta cells, a matrix including one or more of the foregoing, or any combination thereof. Further, a matrix may comprise isolated islet cells, isolated cells from pancreas, isolated cells from a tissue, stem cells, stem cell-derived cells (e.g., stem cell-derived islet cells), induced pluripotent cells, differentiated cells, transformed cells, or expression systems, which can synthesize one or more biological products. In some embodiments, the macroencapsulation device comprises a population of stem cell-derived islet cells. In some embodiments, the stem cell-derived islet cells comprise stem cell-derived beta cells, stem cell-derived alpha cells, and/or stem cell-derived delta cells.

To provide sufficient strength and/or rigidity for a macroencapsulation device, the various membranes and frames may be made from materials that are sufficiently stiff to maintain a desired shape of the macroencapsulation device during use. The desired stiffness may be provided via an appropriate combination of a material's Young's modulus (also referred to as an Elastic modulus), thickness, and overall construction which may be balanced with a desired permeability of the device. Appropriate Young's moduli for the various membranes and frames described herein may be at least 10⁵ Pa, 10⁶ Pa, 10⁷ Pa, 10⁸ Pa, 10⁹ Pa, and/or 10¹⁰ Pa. Other appropriate Young's moduli for the various membranes and frames described herein may be used including moduli both greater than and less than these ranges. Ranges between the foregoing Young's moduli are contemplated including, for example, a Young's modulus between or equal to about 10⁶ Pa and 10¹⁰ Pa.

The frame of a macroencapsulation device may be formed from any appropriate biocompatible thermoplastic material. As previously noted, in some embodiments, an appropriate material for the frame may include polyetheretherketone (PEEK). Appropriate materials for the frame may also include, but are not limited to polycarbonate, polyurethane, polyetheretherketone (PEEK), Polyvinyl Chloride (PVC), poly (oxymethylene), poly (methyl methacrylate) (PMMA), thermoplastic polymer based composites, polypropylene, fluorinated ethylene propylene (FEP), low density polyethylene (LDPE), high density polyethylene (HDPE), ultra-high density polyethylene (UHDPE), polycaprolactone, poly (lactide), poly (glycolic acid), poly lactide-co-glycolide, ethylene vinyl acetate copolymer, polyamides, poly (butylene) therephthalate, combinations of the foregoing, and/or any other appropriate thermoplastic material. In addition to the use of a thermoplastic material in a frame, embodiments in which a frame includes a thermoplastic portion configured to be bonded to a membrane and another non-thermoplastic portion are also contemplated as the disclosure is not limited to frames made completely from a thermoplastic material. In some embodiments, an appropriate material for the frame includes polypropylene. In some embodiments, an appropriate material for the frame includes fluorinated ethylene propylene (FEP). In some embodiments, an appropriate material for the frame includes ultra-high density polyethylene (UHDPE). In some embodiments, an appropriate material for the frame includes polycarbonate. In some embodiments, an appropriate material for the frame includes polyurethane. In some embodiments, an appropriate material for the frame includes PVC. In some embodiments, an appropriate material for the frame includes poly (oxymethylene). In some embodiments, an appropriate material for the frame includes poly (methyl methacrylate (PMMA). In some embodiments, an appropriate material for the frame includes thermoplastic polymer based composites. In some embodiments, an appropriate material for the frame includes polypropylene. In some embodiments, an appropriate material for the frame includes LDPE. In some embodiments, an appropriate material for the frame includes HDPE. In some embodiments, an appropriate material for the frame includes polycaprolactone. In some embodiments, an appropriate material for the frame includes poly (lactide). In some embodiments, an appropriate material for the frame includes poly (glycolic acid). In some embodiments, an appropriate material for the frame includes poly lactide-co-glycolide. In some embodiments, an appropriate material for the frame includes ethylene vinyl acetate copolymer. In some embodiments, an appropriate material for the frame includes polyamides. In some embodiments, an appropriate material for the frame includes poly (butylene) therephthalate. In other embodiments, an appropriate material for the frame or portion of the frame may include titanium, graphene, stainless steel, or other appropriate biocompatible material exhibiting sufficient rigidity to function as a frame for the macroencapsulation device.

As used herein, a pose may refer to a combination of a position (i.e., three dimensional position) and orientation (i.e., an angular orientation) of an object. For example, a pose of an elongated shaft disposed within an internal volume of a container may correspond to both the position and orientation of the elongated shaft within the internal volume of the container. In one such embodiment, and as elaborated on further below, a pose of an elongated shaft may correspond to the elongated shaft being positioned at least partially within the internal volume and being oriented in a direction such that a longitudinal axis of the elongated shaft extends substantially parallel to a longitudinal axis of the container.

It should be understood that the above noted materials, parameter ranges, and general description of the construction and/or operation of various components may be used cither individually or in combination with one another with any one of the embodiments of a macroencapsulation implantation device and/or container disclosed herein.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

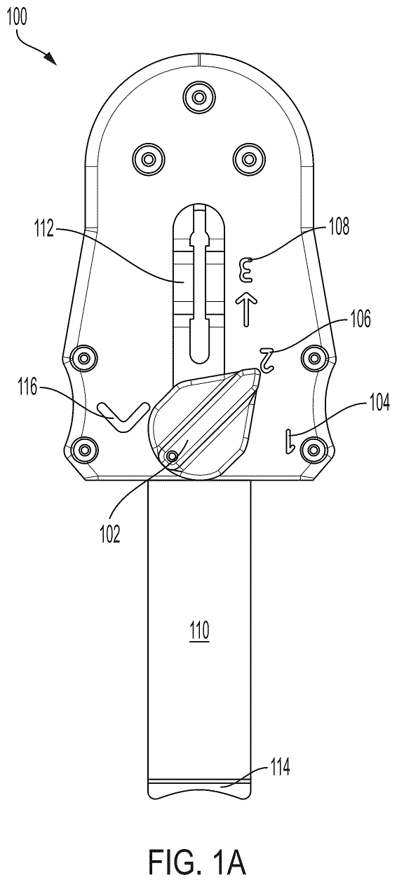

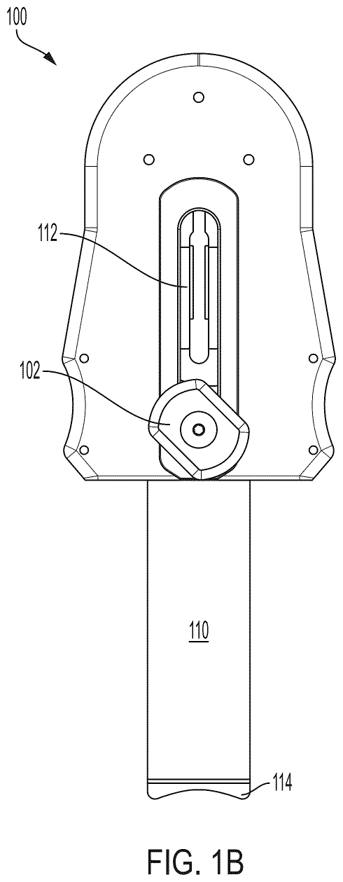

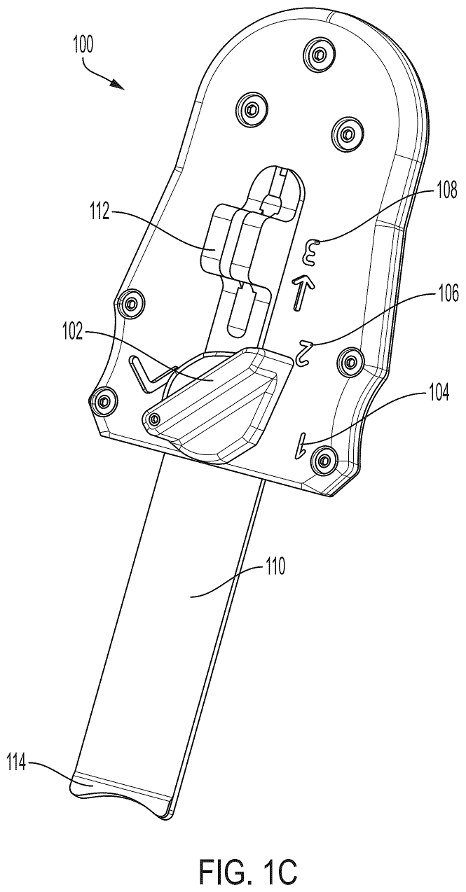

FIGS. 1A-1C show one embodiment of a first portion of a macroencapsulation implantation device including a handle 100 and a pusher 110 extending distally away from the handle. As elaborated on further below the pusher 110 may be sized and shaped to be inserted into an internal channel of a receptacle/elongated shaft, such that a distal portion 114 of the pusher opposite from a portion of the handle that is gripped by a user may be placed into contact with a macroencapsulation device disposed within a recess of the elongated shaft/receptacle. The handle 110 and the elongated shaft may be selectively coupled together by a lock 102. As shown in the figure, the lock may include a knob, or other user actuatable feature (e.g., a latch, switch, button, detent, or other structure) that can be selectively moved between a first configuration 104, a second configuration 106, and/or a third configuration 108. In some embodiments, the lock 102 may include a first portion of the lock located on the elongated shaft/receptacle, and a second portion of the lock located on the handle (e.g., the depicted knob and corresponding locking structure). During operation the handle 100 may be coupled to the elongated shaft by engaging the first portion of the lock and the second portion of the lock together. The lock 102 may be transitioned from the first unlocked configuration 104 to the second locked configuration 106. When the lock 102 is in the second locked configuration 104, the lock 102 or other appropriate trigger may be configured to be displaced to displace the engaged elongated shaft in a proximal direction relative to the handle as shown by the indicated arrow and third configuration 108. In the first configuration 104, an elongated shaft may be disengaged with the handle 100. To disengage the elongated shaft from the handle 100, the lock 102, or other trigger, may be moved to distally displace the elongated shaft back to the initial position relative to the handle 100 and the lock 102 may be moved from the second locked configuration to the first unlocked configuration. The elongated shaft may then be removed from the associated handle 100 including the pusher 110. In some embodiments, switching the lock between the different configurations may offer tactile feedback to a user, such that the user may receive an indication that a change in configuration has occurred in the lock via an audible and/or tactile click. In various embodiments, any appropriate type of lock may be included for selectively coupling/de-coupling the receptacle (i.e., the elongated shaft) and the handle.

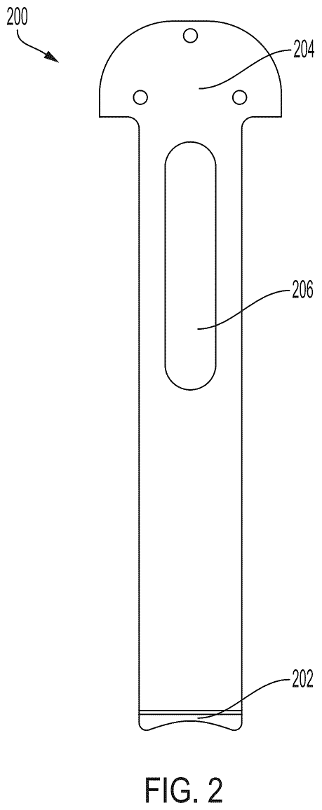

In some embodiments, the macroencapsulation implantation device may also include a safety to prevent unintentional deployment or partial deployment from a macroencapsulation implantation device. In some such embodiments, a safety may prevent the relative movement of an elongated shaft/receptacle and a handle of a macroencapsulation implantation device in a first configuration and may permit the relative movement of the elongated shaft and the handle in a second configuration. A safety may correspond to any appropriate construction capable of selectively permitting or preventing the relative movement of the elongated shaft including, but not limited to, switches, clips, pins, and/or any other component that is capable of selectively blocking or permitting the movement of a structure associated with movement of the elongated shaft relative to the handle. For example, FIGS. 1A-1C illustrate the use of a safety 112 in the form of a clip that is disposed within a slot that the knob of the lock 102 moves through during operation of the elongated shaft (e.g., see elongated opening 206 formed in the pusher 200 in FIG. 2). The presence of the clip within this elongated opening physically blocks movement of the lock 102, and thus, prevents movement of the elongated shaft relative to the handle 100. Once the safety is moved to the second configuration (i.e., it is removed from the elongated opening in this embodiment), the lock 102 or other trigger may be used to move the elongated shaft relative to the handle.

In view of the above, in some embodiments, a safety may be selectively attachable to the handle, and may be configured to selectively prevent the elongated shaft/receptacle from moving relative to the handle by preventing the lock from being displaced in the distal direction while in the second configuration. For example, the safety may obstruct a pathway for the displacement of the lock in the second configuration. As such, until the safety is removed or otherwise moved from a position obstructing the pathway of the lock by a user, the lock and connected elongated shaft may not be displaced.

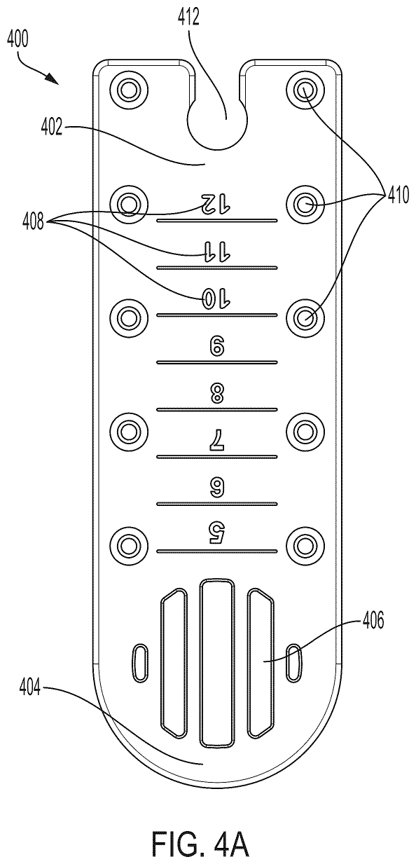

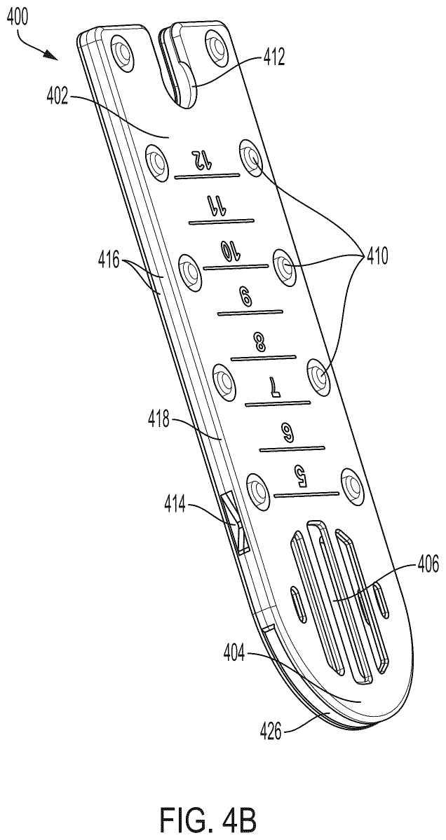

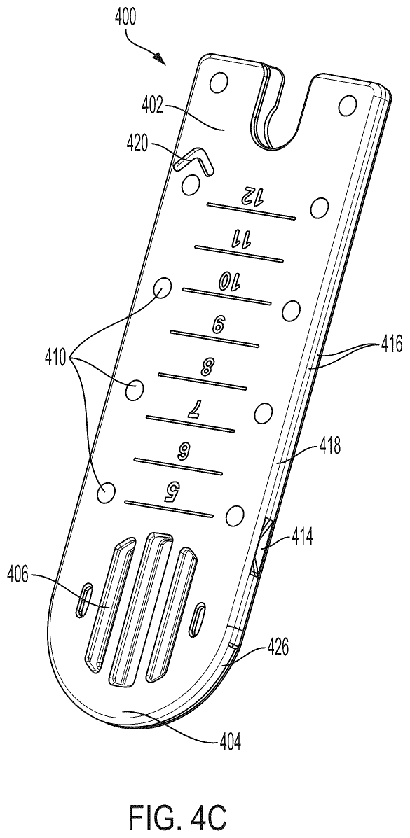

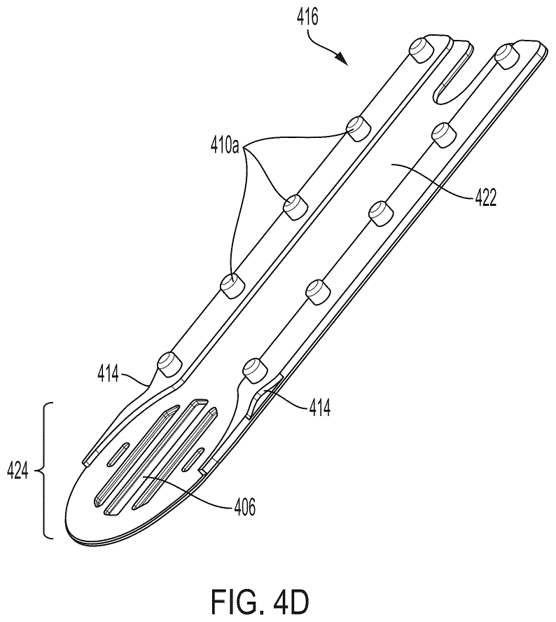

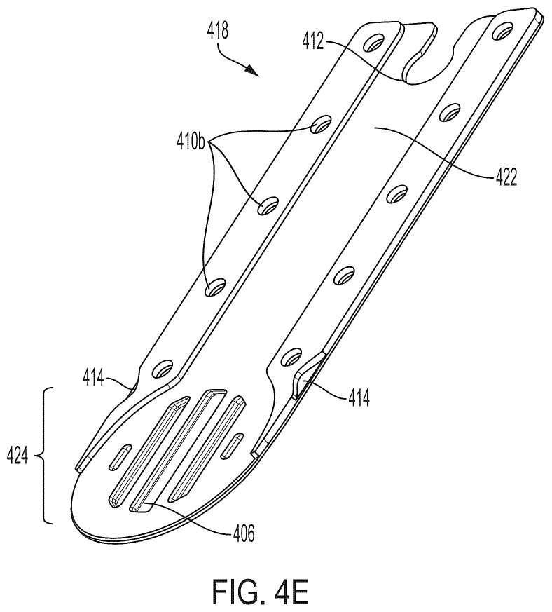

In some embodiments, it may be desirable to indicate a proper orientation of an elongated shaft/receptacle relative to a handle. This may be done using keyed geometries and/or indicators such as the depicted indicator arrow 116 formed on the handle 100 which may include a corresponding indicator. These indicators may be aligned by a user to help assist in connecting an elongated shaft to the handle 100 in the correct orientation (e.g., see corresponding indicator 420 formed on the elongated shaft in FIG. 4C).

In the above embodiment, the movements of the lock 102 between the first configuration and the second configuration, and the movement from the second configuration to the third configuration are shown as a rotation of a knob followed by a proximal displacement along a pathway blocked by a safety 112. However, it should be understood that any desired combination of movements may be used to move a lock between a first unlocked configuration and a second locked configuration. Additionally, while a proximal displacement of the lock is depicted, other methods for displacing the lock and associated elongated shaft may be used. For example, a change between locked and unlocked configurations may be caused by pressing a button, a pathway between the second configuration and the third configuration may be curved, or other appropriate methods for locking and/or displacing these components may be used as the disclosure is not limited to specifically how the components are selectively locked together or displaced.

FIG. 2 shows one embodiment of a pusher 200 which may be attached to a portion of the handle configured to be gripped by a user. For example a proximal portion 204 of the pusher may be configured to be attached to first portion of a handle (e.g., the handle of FIG. 1). The pusher may be attached to a portion of the handle using fasteners, adhesives, welding, brazing, or other appropriate types of connections. Alternatively, the pusher may be integrally formed with the overall handle. The pusher may include an elongated linear portion including a distal end portion 202 of the pusher that is configured to be placed against a macroencapsulation device during use. The pusher 200 may also have an elongated opening 206 formed along at least a part of a length of the pusher 200. The elongated opening may be configured to engage with a portion of the lock to guide and limit movement of the lock, and a connected elongated shaft, relative to the other portions of the handle during proximal and distal movement of the elongated shaft.

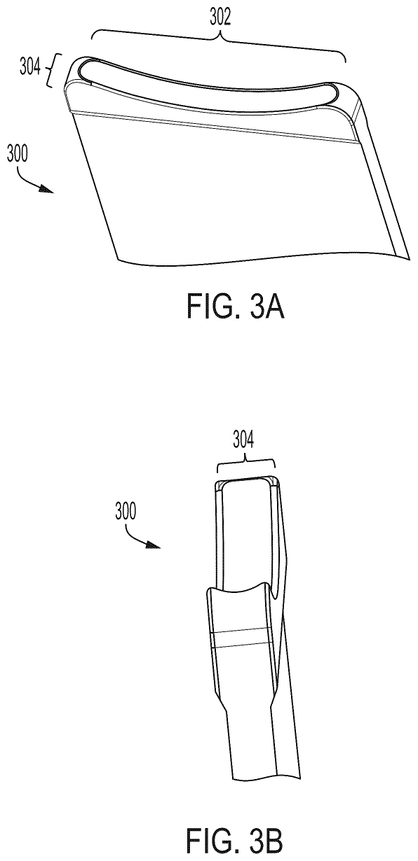

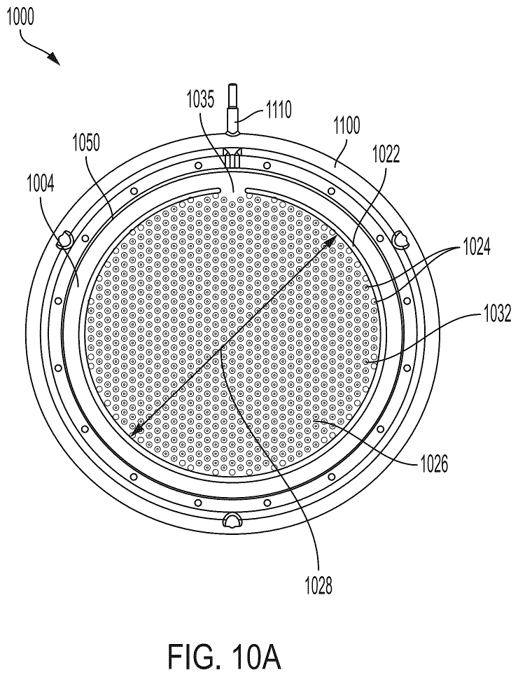

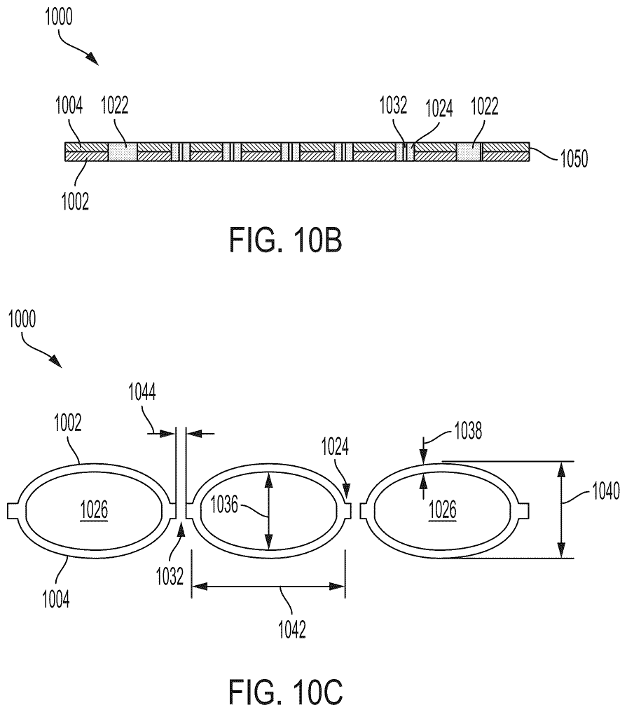

FIGS. 3A-3B show perspective views of one embodiment of a distal portion 300 of a pusher which may be used with the embodiments described above. In some embodiments, the distal portion 300 may be sized and shaped to complement a shape of corresponding portion of a macroencapsulation device disposed within a recess of the elongated shaft the pusher is in contact with. By complementing a shape of the macroencapsulation device with a distal portion of the pusher, a contact area between the pusher and the macroencapsulation device may be maximized while helping to avoid stress concentrations. This may help to spread a more uniform stress across the engaged area of the macroencapsulation device. For example, the distal portion 300 shown in FIGS. 3A-3B may be curved in multiple directions and/or may be chamfered on one or more edges (or optionally all edges that may contact the macroencapsulation device). In the depicted embodiment, the distal portion is curved to complement a circular macroencapsulation device and includes rounded edges. A width 302 of the distal portion 300 may have a radius of curvature capable of conforming at least in part to a radius of curvature of a macroencapsulation device. A thickness 304 of the distal portion 300 may also have a radius of curvature capable of conforming at least in part to rounded and/or chamfered edges of the macroencapsulation device. An example of a macroencapsulation device which may be used with the pusher 300 is described in further detail below relative to FIGS. 10A-10C. Depending on the embodiment, the curvature in the width direction of the pusher may be different from a curvature in the thickness direction of the pusher. Additionally, as shown in more detail below relative to FIGS. 4A-4E, a width of the pusher may be less than the width of the transverse cross section of the recess, not depicted in FIGS. 3A-3B.

FIGS. 4A-4E show an elongated shaft 400 which may be removably attached to a handle using a second part of a lock 412. FIG. 4A shows a view of a first side of the elongated shaft 400, with a proximal end portion 402 and a distal end portion 404, wherein at least a portion of the proximal end portion 402 may be engaged with a handle. Specifically, in the depicted embodiment, the second part of the lock 412 is a shaped slot including a neck and an associated interior opening with a width that is larger than a corresponding width of the neck. This forms the second part of a cam lock, where a first portion of the lock is rotated into the second configuration where a dimension of the first portion of the lock is too large to pass through the neck of the slot, thus locking the handle and elongated shaft together, though other types of locks may also be used. The distal end portion 404 of the elongated shaft may include a recess 424 formed therein which is configured to hold a macroencapsulation device disposed within the recess. The macroencapsulation device can enter and exit the recess 424 through a distal opening 426 formed in the distal end portion of the elongated shaft. The distal opening may have a transverse cross section perpendicular to a longitudinal axis of the elongated shaft that is sized and shaped to accommodate passage of the corresponding transverse cross section of the macroencapsulation device. For example, in the depicted embodiment, a width and thickness of the transverse cross section of the distal opening may be greater than a corresponding width and thickness of the macroencapsulation device. In some embodiments, a distal portion of the elongated shaft/receptacle may be shaped to complement a shape of the macroencapsulation, e.g., the distal end portion 404 of elongated shaft 400 comprises an end shaped as a semicircle to conform to a circular macroencapsulation device contained within a circular recess. However, such conformity is not required, and the distal portion may comprise any suitable geometry as long as the elongated shaft/receptacle can retain a macroencapsulation device within a recess formed in the distal end portion of the elongated shaft.

The elongated shaft 400 may include an internal channel 422 which extends from a proximal end portion of the elongated shaft including an opening an associated pusher is inserted through to the recess 424 formed a distal portion of the elongated shaft. The interior channel may extend parallel to, and in some instances along, a longitudinal axis of the elongated shaft. Again, the recess may form a distal opening of the internal channel in the distal end portion of the elongated shaft. A maximum width of a transverse cross section of the recess and/or the associated opening in a first direction perpendicular to the longitudinal axis of the elongated shaft may be greater than a thickness of the transverse cross section of the recess in a second direction perpendicular to the longitudinal and the first direction in some embodiments. Additionally, in some embodiments, a width of the internal channel 422, and the corresponding pusher configured to be disposed therein, may be less than a corresponding maximum width of the recess and/or distal opening of the elongated shaft. During use a pusher of the handle may be inserted through an internal channel 422 of the elongated shaft 400 as the handle and the elongated shaft are connected together, and a width and thickness of the pusher may be sufficiently smaller than a width and thickness of the internal channel of the elongated shaft to permit a slip fit of the pusher within the internal channel.

In the depicted embodiment, the internal channel 422 of the elongated shaft 400 has a rectangular transverse cross sectional shape perpendicular to a longitudinal axis of the elongated shaft. However, other shapes including squares, rounded rectangles, elongated ovals, and/or any other shape capable of passing a pusher for engagement with a corresponding macroencapsulation device may be used as the disclosure is not so limited. Also, while a circular macroencapsulation device is depicted, other macroencapsulation devices with other cross sectional shapes such as squares, rectangles, ovals, or other appropriate shapes may be used as the disclosure is not limited to the shape of the macroencapsulation device, the recess it is received in, or the opening through which it is deployed. The depicted recesses and macroencapsulation device exhibit widths that are significantly larger than their thicknesses such that the depicted recess and macroencapsulation devices configured to be received therein may have flat plate like geometries in some embodiments. In either case, a size and shape of a recess formed in the distal end portion of an elongated shaft/receptacle may be sized and shaped to at least partially complement a size and shape of a macroencapsulation device disposed within the recess.

In some embodiments, the elongated shaft/receptacle may be a two-piece construction. This may include a first section 416 and a second section 418 of the elongated shaft 400 that may be connected along their lengths at connections 410. For example, the male connectors 410a of the first section 416 may be connected to the female connectors 410b of the second section 418 through heat staking, threaded fasteners, a friction fit, rivets, adhesives, brazing, or other appropriate method. During heat stacking, the male connectors may be melted and/or deformed after being inserted through the female connectors to permanently couple the two sections together, though as noted above other types of connections may also be used. In some embodiments, the edges and/or seams of the elongated shaft may be rounded and/or chamfered to help prevent tissue damage upon insertion and removal from a subject. Additionally, in some embodiments, the elongated shaft/receptacle may be made from an integral piece of material using casting, injection molding, machining, combinations of the forgoing, and/or any other appropriate manufacturing method as the disclosure is not so limited.

As noted previously, a plurality of slots 406 may be formed in a distal end portion 404 of both a first section 416 and a second section 418 of the elongated shaft 400. Accordingly, the plurality of slots may include a first group of slots formed on a first surface of the distal portion of the elongated shaft 400 and a second group of slots formed on a second surface of the distal portion of the elongated shaft 400 opposite from the first surface. These slots may be aligned with and formed in a portion of the elongated shaft including the recess 424. The slots may be elongated linear slots, or other appropriately shaped slots, as previously described above. Thus, the slots may provide fluid communication between the recess 424 and an external environment surrounding the elongated shaft 400. Thus, this may expose a macroencapsulation device disposed within the recess 424 to an environment surrounding the elongated shaft 400 through the plurality of slots which may be formed on at least one side or on two opposing sides of the recess. In some instances, the slots may be positioned such that they lie within an outer perimeter of a shape of the recess. For example, as shown best in FIG. 4D, the slots 406 are formed in a portion of the elongated shape forming the recess and are contained within an interior of the circular cross section of the recess, though other geometries may also be used. As also described above, the slots may extend in a direction that is substantially aligned with a longitudinal axis of the elongated shaft/receptacle in some embodiments to help minimize the engagement with and/or abrasion of tissue during insertion of the elongated shaft/receptacle into a target surgical site (e.g., within 5°, or more preferably within 1°). In some embodiments, it may be desirable for the slots to included rounded corners to help minimize abrasion with tissue the slots are moved across during an implantation procedure.

In some embodiments, an elongated shaft/receptacle may be configured with features which may improve the utility and case associated with manipulating and using the macroencapsulation device for a user. For example, the elongated shaft 400 may include length indicators 408 (e.g., distance markings) disposed on an outer surface of the elongated shaft and along a length of the elongated shaft. In one such embodiment, these length indicators 408 may be positioned on the external surfaces of the first section 416 and/or the second section 418 on the two large opposing surfaces of the elongated shaft. The length indicators 408 may assist a user in estimating the implantation depth of a macroencapsulation device during an operation. In some embodiments, the length indicators may be in increments of any desired length measurement. Furthermore, at least one external surface of a section of the elongated shaft 400 may have an orientation indicator 420 that corresponds to the orientation indicator formed on the handle as described above. By aligning these indicators, it may be possible to properly orient the elongated shaft 400 relative to a handle of a macroencapsulation implantation device.

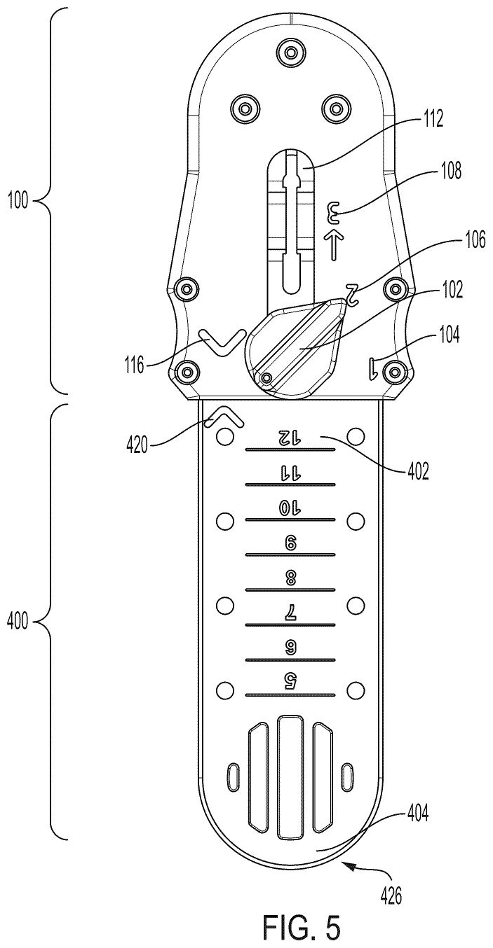

FIG. 5 shows a macroencapsulation implantation device where an elongated shaft 400 is connected to a handle 100 by a lock 102 that has already been moved from the first unlocked configuration 104 to the second locked configuration 106. In this configuration, a proximal end portion 402 may be partially disposed within the handle 100 and the pusher, not depicted, is inserted into and disposed within the correspondingly shaped internal channel of the elongated shaft 400. As previously discussed, the orientation indicators 116 and 420 disposed on the handle 100 and elongated shaft 400 may be aligned with one another indicating that these components are positioned in the correct orientation relative to one another. If a macroencapsulation device is present, it may be disposed within a recess formed within the distal end portion 404 of the elongated shaft as described previously above. Additionally, a distal end portion of the pusher may be disposed adjacent to, and optionally in contact with, the macroencapsulation device within the recess of the elongated shaft 400 when the elongated shaft 400 is connected to the handle 100 and the lock 102 is initially moved to the second locked configuration and prior to the relative displacement of the elongated shaft 400 and handle 100.

When it is desired to displace the macroencapsulation device out of a distal opening of the elongated shaft/receptacle, the depicted safety 112 may either be removed, or otherwise moved to an unlocked configuration to permit movement of the lock 102 in a proximal direction relative to the handle. Thus, the lock 102 may be displaced towards a third configuration 108 from the second configuration 110, and the elongated shaft 400 may be moved in a corresponding proximal direction relative to the handle 100. In one embodiment, the lock may be displaced along a predetermined path in a direction that is substantially parallel to a longitudinal axis of the handle when the lock moves from the second configuration towards the third configuration. For example, the lock may be displaced within an elongated opening 206 formed in the pusher, see FIG. 2, while the pusher is held stationary relative to the other portions of the handle 100. Thus, the elongated shaft 400 may be moved proximally relative to the handle 100 and pusher while the handle 100 and pusher are held stationary relative to a reference frame of a user and/or robotic surgical system interacting with the macroencapsulation implantation device.

As the elongated shaft 400 is displaced proximally relative to the handle 100, and the associated pusher positioned within an internal channel of the elongated shaft, a distal end portion of the stationary pusher may apply a force to the associated macroencapsulation device disposed within the recess to hold the macroencapsulation device stationary relative to the handle. Thus, as the elongated shaft 400 is moved in the proximal direction from an extended configuration to a retracted configuration, the stationary macroencapsulation device may be displaced out of a distal opening 426 formed in the distal end portion 404 of the elongated shaft. In some embodiments, the fully retracted configuration of the elongated shaft 400 and the lock 102 may correspond to the third configuration of the lock 102.

After deployment of the macroencapsulation device from the distal opening 426 of the elongated shaft 400, the macroencapsulation implantation device may be removed from a deployment site within a subject. Optionally, the lock 102 may be moved from the third configuration 108 back the second configuration 106 to move the elongated shaft 400 from the retracted configuration back to the extended configuration relative to the handle 100. The lock may then be moved from the second locked configuration 106 to the first unlocked configuration 104. The elongated shaft 400 may then be disengaged and removed from the handle 100. With the elongated shaft 400 released from the handle 100, the handle 100 may optionally be used to engage with and deploy another macroencapsulation device disposed within another elongated shaft.

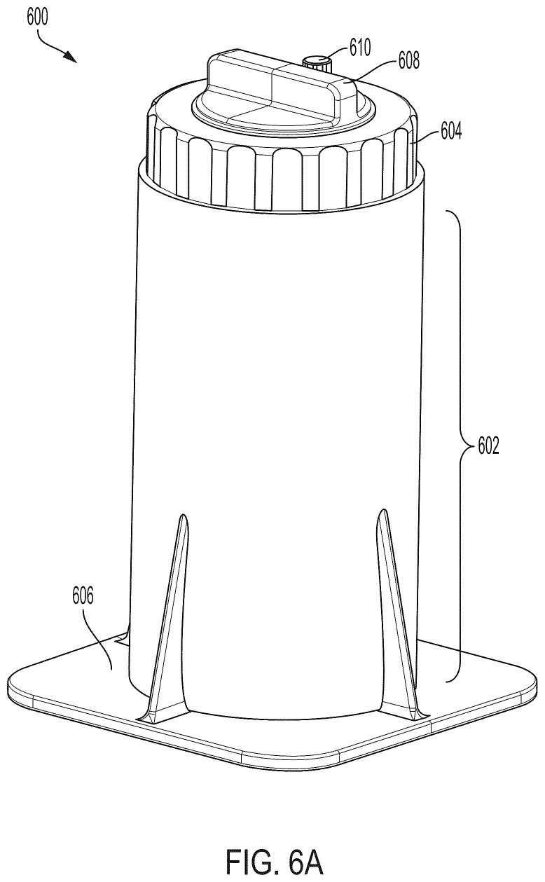

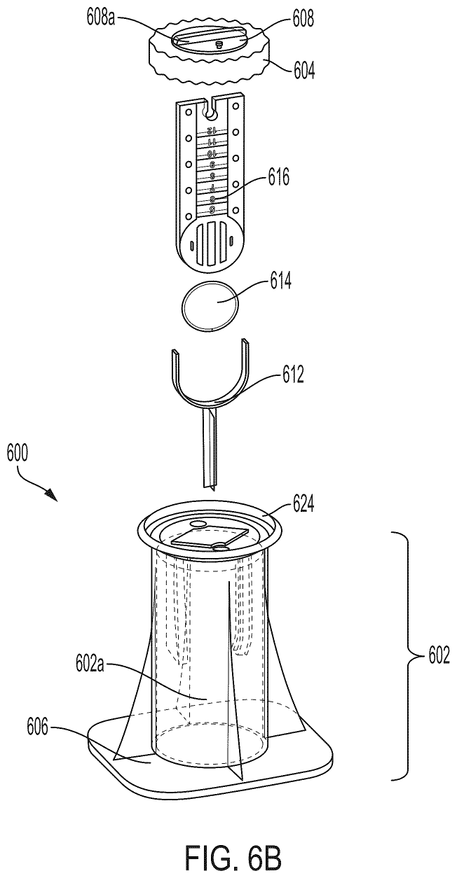

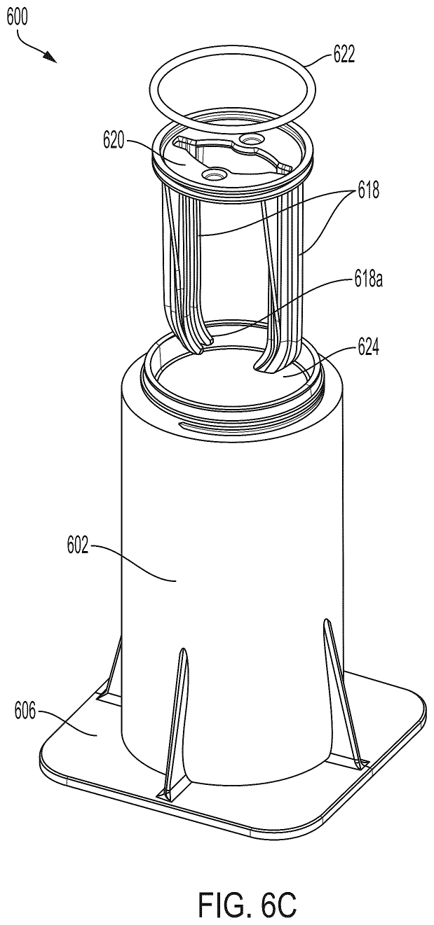

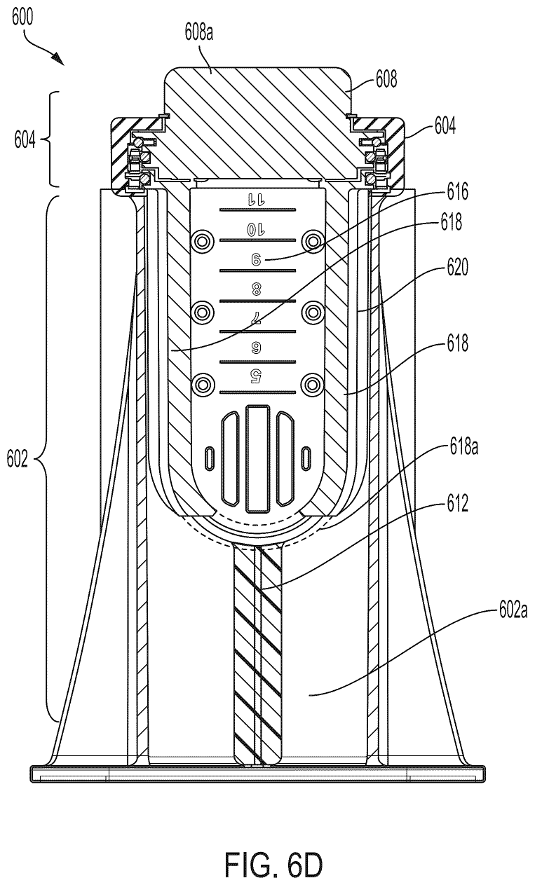

FIGS. 6A-6D show various views of one embodiment of a container 600 which may be used to store a macroencapsulation device 614 positioned within an internal channel and/or recess of an elongated shaft 616 of a macroencapsulation implantation device. This includes the illustration of the different internal components of the container 600 which may be configured to support the elongated shaft 616, and the associated macroencapsulation device, in a predetermined pose within an internal volume 602a of the container 600. Again, this may help to both protect the macroencapsulation device and provide appropriate exposure to a surrounding media when the macroencapsulation device is stored within the container prior to use.

A body 602 of the container 600 may include an internal volume 602a that extends from an opening 624 in the body into an interior of the body. The internal volume 602a may be appropriately sized and shaped to at least partially receive an elongated shaft 616 of a macroencapsulation implantation device therein as well as other internal components of the container 600 which may be configured to support the elongated shaft 616, and the macroencapsulation device 614 disposed therein, in a predetermined pose. When the elongated shaft 616 is properly positioned within the internal volume 602a of the container 600, the macroencapsulation device 614 may be in fluid communication with a media contained within the internal volume 602 of the body through a plurality of slots on the elongated shaft 616 as previously described.

The container 600 may include any number and/or type of supports to maintain the elongated shaft 616 in the predetermined pose. For example, as best seen in FIGS. 6C and 6D, opposing portions of the elongated shaft 616 (e.g., opposing thin side surfaces extending between the larger flat surfaces of the depicted elongated shaft) may be slidably engaged with opposing grooves formed in and extending along at least a portion of a length of a pair of rails 618. The pair of rails 618 may extend into the internal volume 602a of the container 600 and may be positioned on opposing sides of a longitudinal axis of the internal volume 602a of the body 602 of the container 600. A groove 618a may extend along at least a portion of a longitudinal length of each rail of the pair of rails. Thus, the elongated shaft may be engaged with the opposing pair of grooves such that the elongated shaft 616 is engaged with and retained in the grooves 618a between the two opposing rails 618 in the desired predetermined pose in the internal volume. In some embodiments, the pair of rails 618, and the associated grooves may be configured such that a gap between the pair of rails may be larger proximate to the opening 624 of the internal volume 102a relative to a distal portion of the pair of rails 618 spaced from the opening 624. For example, the rails and associated grooves may curve inwards as they extend in a longitudinal direction as shown in the figures with a shape that conforms to a corresponding size and shape of the elongated shaft. For example, the distal inner portion of the rails 618 and grooves 618a located opposite from the opening 624 may optionally be sized and shaped to partially surround a distal end portion of the elongated shaft which may help to maintain a desired spacing of the elongated shaft away from a bottom surface of the interior volume located opposite from the opening.

It should be understood that the rails 618 of a container may be formed in any appropriate portion of a container. In the depicted embodiment, the rails are formed as a portion of an intermediate support 620. The intermediate support includes a first portion that is configured to be attached to a portion of the container proximate the opening 624 or other portion of the container such that the rails 618 may be supported and extend from the first portion of the intermediate support into an interior of the internal volume 602a in a predetermined pose. In some instances, the intermediate support may be a plate like structure that is configured to be disposed on and rotatably slide on a supporting surface of the body 602. However, the use of non-rotatable supports and/or other types of attachments are contemplated including integral formation of the intermediate support 620 and rails 618 with the body 602 and/or attachment of the intermediate support 620 to the body 602 using connectors such as threaded fasteners, welds, adhesives, mechanically interlocking features, and/or any other appropriate connection method for supporting the intermediate support 620 and rails 618 relative to the interior volume 602a of the container 600. In some embodiments, the pair of rails and (optionally) their respective grooves may be connected at their distal ends, such that the pair of rails may be a single continuous structure (though each rail may be formed as separate components and joined to form the single continuous structure). For example, in some embodiments, the pair of rails 618 may optionally be joined by a rail connector 618a. In some embodiments, the rail connector 618a may additionally connect the grooves of the rails 618, though in other embodiments the rail connector may comprise one or more flat walls connecting only the outer surfaces of each rail 618.

In some instances, it may be desirable to prevent leakage between the intermediate support 620 and the opening 624 of the body 602 of a container 600. In such an embodiment, a seal 622 may be disposed between a portion of the intermediate support and another portion of the container 600, such as a corresponding cap 608 and/or the body 602 of the container. For example, in some embodiments, a seal 622 may be present between an outer rim of the intermediate support 620 and the cap 608 of the container. The seal 624 may help to seal the internal volume relative to a surrounding environment which may help prevent contaminants from entering the container and/or prevent media and/or fluid within the container from leaking out of the container. The seal 624 may be at least one of an O-ring, flange seal, extruded profile, static radial seal, axial face axial seal, and any other suitable type of seal. Appropriate materials for the seal may include silicone, rubber, and/or any other suitable clastic material.

To help facilitate manipulation and engagement of an elongated shaft with a corresponding handle, in some embodiments, it may be desirable for a proximal portion of an elongated shaft 616 to extend proximally out of the opening 624 of the container 600. For example, by having a portion of the elongated shaft extending in a proximal direction out of the opening 624 when the elongated shaft 616 is positioned in the container 600, it may facilitate the manipulation and engagement of the proximal portion of the elongated shaft 616 with a handle of a macroencapsulation implantation device. Thus, the rails 618 and/or other supports may be configured to support the elongated shaft 616 in a pose with the proximal portion of the elongated shaft 616 extending out of the opening. In such an embodiment, it may be desirable to have a cap 608 that is configured to engage with and help maintain the elongated shaft 616 in the desired pose. For example, the container may include a cap 608 which may include a protrusion 608a extends outwards, e.g., along a longitudinal axis of the internal volume 602a, in a direction away from the body 602 and opening 624 of the container 600 when the cap 608 is attached to the body 602 of the container 600. The protrusion 608a of the cap may include a cavity that is oriented towards the interior volume 602a when attached to the container body 602. The cavity of the protrusion may be sized and shaped to receive a proximal end portion of the elongated shaft 616 disposed therein when the cap is positioned on the container body 602. This may help with retaining the elongated shaft/receptacle in a desired pose within the internal volume 602a of the container by retaining the elongated body between the protrusion 608a of the cap 608 and the rails 618.

In some applications, it may be desirable to sample a media contained within the container. Thus, in some embodiments, the cap may include a sampling port 610 which may be used for sampling a media contained within the body 602. In some embodiments, the sampling port 610 may be a luer port which prevents any undesired exchange of fluid from an external environment of the container 600 and an internal volume 602a of the container 600. Accordingly, in some embodiments, the container cap includes a sampling port 610. In some embodiments, the container cap does not include a sampling port 610. In some embodiments, the sampling port may be used to exchange media within a container.

The cap 608 may be releasably coupled to the body 602 of the container 600 using any appropriate type of releasable attachment including, but not limited to, mating threads formed on the cap 608 and container body 602, threaded fasteners, clamps, interference fits, mechanically interlocking features, and/or any other appropriate type of releasable attachment. In some applications, it may be desirable to maintain the elongated shaft/receptacle stationary relative to the container body during opening. In such an embodiment, a rotational clamp 604 may include threads that engage corresponding threads formed on the body 602 such that the rotational clamp may compress the cap 608 against the container body 602. During use, the protrusion of the cap 608 may be grasped by a user to hold the cap and container body stationary relative to each other. The rotational clamp 604 may then be rotated relative to the cap and container body 602 to remove the cap. This may aid in maintain the elongated shaft 616 stationary during opening of the container and avoid the application of movements and/or forces to the macroencapsulation device.

During use, the cap 604 may be removed from its engagement with the body 602. After the cap 604 has been removed, a handle with a lock in the first configuration may be engaged with a proximal portion of the elongated shaft 616 extending out of the opening 624 of the container. As the elongated shaft 616 may protrude from an opening 624 of the body 602, a user may easily engage the handle and the elongated shaft 616 together. After the two parts are connected, the lock on the handle is selectively moved to a second configuration, as described previously above, such that the elongated shaft 616 and the handle may be connected. The handle and elongated shaft assembly may then be removed from the container as a single assembly whenever the user is ready to implant the macroencapsulation device disposed within a recess of the elongated shaft.

As previously described, an internal volume 602a of a container 600 may be designed to facilitate diffusion of oxygen, nutrients, and waste between a media contained in the internal volume and a macroencapsulation device 614 disposed within the elongated shaft 616. As such, in some embodiments, the container 600 may be configured with dimensions and/or geometries that are appropriate to immerse at least a distal end portion of the elongated shaft/receptacle where the macroencapsulation device is disposed within an appropriate volume of media. The volume of media, and the gaps present between a wall of the internal volume 602a and elongated shaft may be sufficiently large to ensure appropriate diffusion of waste and nutrients between the media in the internal volume and the macroencapsulation device. For example, the body 602 may be an elongated cylinder with a body 602 and opening 624 that are sized and shaped to accept the elongated shaft 616 therein. However, the use of an elongated cylinder may result in an unstable container that does not easily maintain its orientation on a supporting surface during use. Accordingly, the container 600 may include a base 606 connected to a bottom portion of the container body 602a that is configured to support the container 600 on a supporting surface. To improve the stability of the container 600, the base may have a transverse cross sectional area that is larger than a transverse cross sectional area of the body 602 of the container. Accordingly, the base may be appropriately sized to help provide stability for maintaining a desired pose of container 600 on a supporting surface. Additionally, in some embodiments, struts or other reinforcement features may extend between an outer surface of the body 602a and the base 606 to improve the stability and rigidity of the container 600.

In addition to supporting an elongated shaft 616, and macroencapsulation device disposed therein, in a desired pose within a container 600, it may be desirable to help prevent unintentional movement of the macroencapsulation device out of a distal opening of the elongated shaft while it is disposed within the container 600. Accordingly, as best shown in FIG. 6B, a cover 612 may be configured to be selectively engaged with and cover at least a portion, or the entire, distal opening of the elongated shaft 616 while the elongated shaft 616 is disposed within the container 600. By obstructing the distal opening of the elongated shaft/receptacle, the macroencapsulation device may be prevented from unexpectedly moving out of the recess and distal opening. The cover may be selectively attached to, and removed from, a distal portion of the elongated shaft 616 using any appropriate type of connector. For example, detents 414 located on opposing sides of a distal portion 404 of the elongated shaft on opposing sides of the distal opening 426, see FIGS. 4A-4E, may be selectively engaged with corresponding detents 704 disposed on a corresponding portion of the cover 612, see FIG. 7. However, in some embodiments, the detents may be located on different portions of the cover and/or elongated shaft/receptacle. Additionally, other selectively attachable and removable connections between the cover and elongated shaft/receptacle may be used including, for example, friction fits, magnetic connectors, clamps, mechanically interlocking features, threaded fasteners, and/or any other appropriate type of removable connection.

FIG. 7 shows a cover 612 in closer detail. The cover 612 may be composed of an elastically deformable material, such that cover 700 may elastically deform until the protrusions of detents 704 are engaged with detents on the elongated shaft. In some embodiments, the cover 612 may additionally include one or more protrusions 706 that are configured to extend at least partially into a distal opening of an associated elongated shaft when the cover is disposed on a distal portion of the elongated shaft. These one or more protrusions in the depicted embodiment extend in a proximal direction away from a surface of the cover that is oriented towards the elongated shaft and distal opening when the cover is attached to the elongated shaft such that the one or more protrusions extend into the opening. The protrusions may help to maintain a macroencapsulation device in a desired pose within a recess of the elongated shaft. The one or more protrusions 706 may be configured to complement and/or conform to the dimensions and/or geometry of the macroencapsulation device to help minimize the forces and/or pressure applied to the macroencapsulation device while also restricting the movement of the macroencapsulation device disposed within the elongated shaft.

While the rails of a container may help to maintain an elongated shaft/receptacle in a desired pose within the container, it may be desirable to include additional support to maintain a desired positioning of the elongated shaft/receptacle relative to a bottom surface of a container. For example, as shown in FIGS. 6B, 6D, and 7, in some embodiments, a cover 612 that is selectively engageable with a distal portion of an elongated shaft 616 may comprise a support shaft 702 which assists the elongated shaft in maintaining a pose within the container. A support shaft may extend away from the cover in a distal direction oriented away from the elongated body 616 and towards the bottom surface of the internal volume 602a of the container when the cover 612 and elongated shaft are disposed in the internal volume 602a. Thus, the support shaft may extend distally away from the distal portion of the elongated shaft when the cover is positioned on the elongated shaft. The length of the support shaft may be selected to maintain a desired spacing between the distal portion of the elongated shaft and the bottom surface of the internal volume 602a of the container opposite the opening 624. In some embodiments, the support shaft 702 may contact a bottom surface of the internal volume 602a of the container. Additionally, during manipulation and use of the macroencapsulation implantation device and the associated container by a user, the support shaft 702 may serve as a visual reminder to remove the cover 612 prior to any attempts to implant the macroencapsulation device.

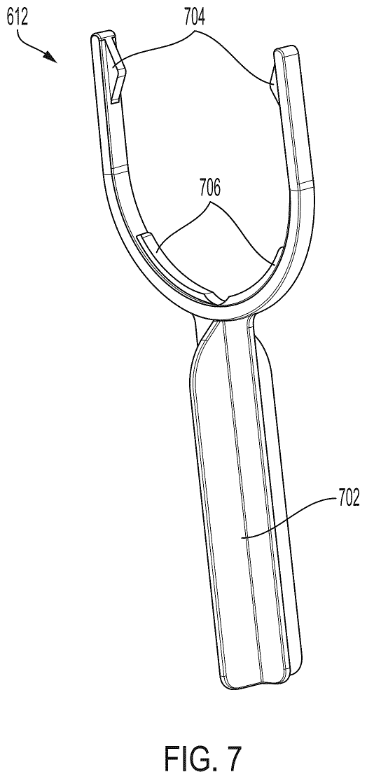

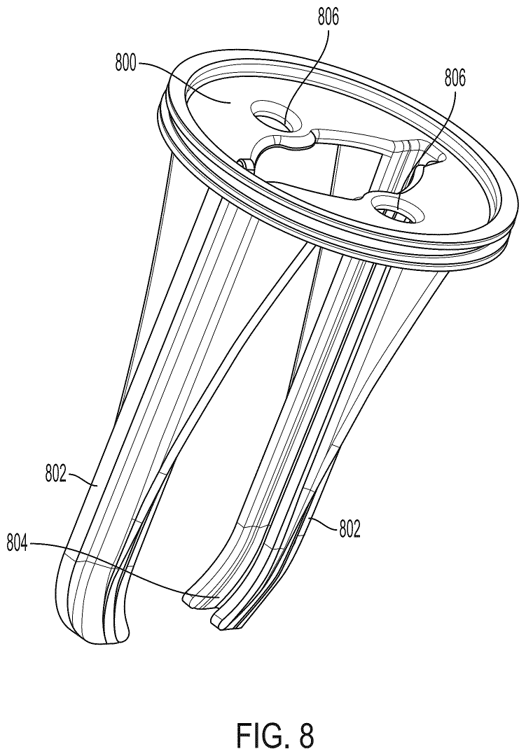

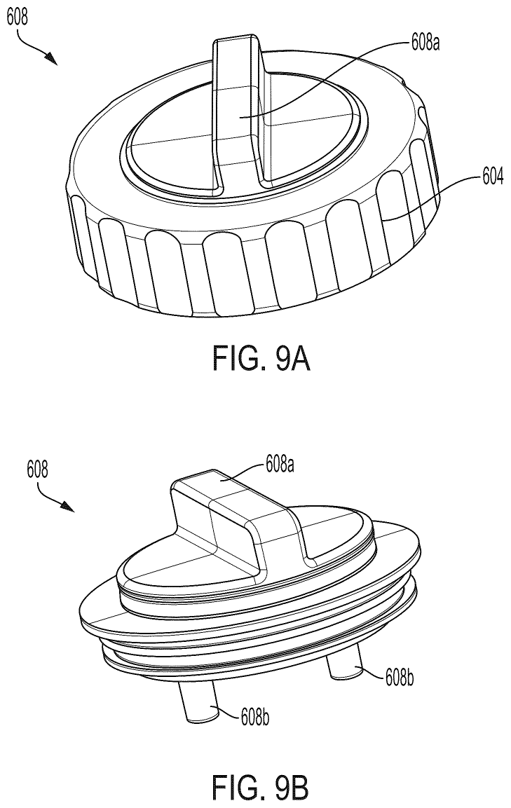

FIG. 8 shows an intermediate support 800 and an associated pair of rails 802 extending away from the intermediate support 800. As previously shown, the pair of rails 802, and groove 804 within each rail, may be positioned on opposing portions of the intermediate support 800 such that groves 804 formed in the rails 802 may be oriented towards each on opposite sides of a longitudinal axis of the intermediate support 800. In addition to this, the intermediate support 800 may include coupling holes 806, or other type of connector, formed in a body of the intermediate support 800, which may mate with corresponding connectors formed on a cap of the container, as shown in FIG. 9B described below. For example, the holes 806 may be sized and shaped to receive protrusions 608b extending from an inner surface of the cap 608. The resulting coupling between the intermediate support 800 and the cap 608 of the container may prevent rotation between the cap and intermediate support, as well as the elongated shaft engaged within the grooves 804 of the intermediate support 800. This may help avoid the application of undesirable rotational forces to the elongated shaft and macroencapsulation device when the cap 608 is removed from the container.

FIGS. 9A-9B show a cap 608 of a container, which may be removably coupled to a body of the container as described previously above. The cap 608 may again include a threaded rotational clamp 604 which may clamp the cap against a body of the container. The rotational clamp 604 may include a threaded connection, which may be coupled with a corresponding threaded connection on an opening of the container. In FIG. 9B, the cap 608 is depicted without the outer rotational clamp 604. As seen in this figure, the cap 608 may include one or more couplings configured to engage with the intermediate support of a container in the form of one or more protrusion 608b, which as described above, may be configured to be selectively engaged with and removed from the coupling holes 806, or other type of selectively removable connection, of the intermediate support as shown in FIG. 8.

FIGS. 10A-10C show an embodiment of a macroencapsulation device 1000 which may be used with any one of the embodiments of a macroencapsulation implantation device and/or container systems disclosed herein. As shown in the figures, a macroencapsulation device may comprise a first membrane layer 1002 and a second membrane layer 1004. In some embodiments, the first and/or second membrane layer may comprise a polymer material, such as ePTFE. In various embodiments, each of the first membrane layer 1002 and the second membrane layer 1004 may be either sintered or unsintered. Each of the first and second membrane layers may also comprise a single layer or multiple layers.

The first and second membrane layers 1002, 1004 may be bonded together at a bonded perimeter 1022 and bonded portions 1024 located within the bonded perimeter. In FIG. 10A, a top surface of the second membrane layer 1004 is shown with a bonded perimeter 1022 of the membranes (e.g., where first and the second membrane layers are bonded) extending around a perimeter of the membranes. The bonded perimeter 1022 may form an interior volume disposed between the first and second membrane layers configured to encapsulate a population of cells. In some embodiments, the bonded perimeter 1022 may extend entirely around the perimeter of the membrane; however, as shown in FIG. 10A, the bonded perimeter may have an unbonded portion 1035, for example to accommodate and/or cooperate with a fill port through which a population of cells may be introduced into an interior volume of the device. As will be appreciated, the dimensions of the bonded perimeter 1022 may at least partially define a size of the interior volume. For example, in embodiments in which the membrane(s) and/or device have a generally circular shape, the bonded perimeter 1022 may have a diameter 1028, which may at least partially define a size of an interior volume between the first and second membrane layers.

In some embodiments, an interior volume between the first and second membrane layers may include a network of continuous interconnected volumes formed by and/or between the various bonded portions of the membrane. For example, as shown in FIG. 10C, an interior volume between the first membrane layer 1002 and the second membrane layer 1004 may include a network of interconnected volumes (e.g., channels 1026) formed between the bonded portions 1024. In some embodiments, an interior volume may have a volume thickness 1036, which may be a maximum distance between the first and second membrane layers in a direction perpendicular to a maximum transverse dimension of the device. As will be appreciated, a total thickness of the membrane (e.g., the total thickness 1040), may depend at least in part on the internal volume thickness 1036, as well as the membrane layer thickness 1038 for each membrane layer. As described herein, a thickness may be measured in a direction perpendicular to a maximum transverse dimension of the device.

As shown in the figures, the bonded perimeter may be disposed radially inward from the outer perimeter 1050 of the membranes. The bonded portions 1024 may take the form of bonded dots distributed across a surface area of the membranes in a hexagonal array.

However, any appropriate shape, arrangement, configuration, and/or spacing of these bonded regions may also be used. For example, as shown in FIG. 10C, a bond spacing 1042 may be a distance between adjacent bonded portions 1024. Additionally, in some embodiments, one or more bonded portions 1024 may include through holes 1032 formed therein. Each through hole may have a through hole diameter 1044. Due to the presence of these bonded regions located radially inwards from a bonded perimeter of the membranes, an internal volume formed between the membranes, once in the filled configuration (e.g., as shown in FIG. 10C), may take the form of a plurality of interconnected channels 1026 corresponding to the unbonded regions of the membranes extending between these bonded portions.

In some embodiments, when the membrane layers are connected to a frame 1100 that extends at least partially and, in some embodiments entirely around, the bonded perimeter 1022 of the membranes. The unbonded portion 1035 of the membranes may be positioned and scaled around a fill port 1110 of the frame such that the fill port remains in fluid communication with the interior volume. In some embodiments, a fill port 1110 may be included in the frame 1100 to allow fluid communication in at least one direction between an external environment and the interior volume of the device. For example, a fill port 1110 may be configured to allow the population of cells to be introduced into the volume between the first and second membrane layers 1002 and 1004. The fill port 1110 may include a through hole, not depicted, extending through the fill port to an interior volume of the macroencapsulation device 1000 formed by the first and second membrane layers 1002 and 1004 shown in FIGS. 10A-10C above.

In various embodiments, a frame 1100 may be formed in any appropriate shape, including any appropriate round, elongated, rectilinear, polygonal (e.g., pentagonal, hexagonal, octagonal, etc.), and/or any other appropriate regular or irregular shape. For example, in the embodiment shown, the frame 1100 may be formed in a generally circular shape. A frame thickness may be a maximum thickness between any two opposing surfaces or points of a cross-section of the frame. In some embodiments, a frame thickness may be measured in a direction perpendicular to a maximum transverse dimension of the frame 1100 and/or the device 1000.

While the above embodiment of a macroencapsulation device may be used with any of the macroencapsulation implantation devices and/or containers, it should be understood that the various embodiments disclosed herein are not limited to being used with such a device. Instead, the various embodiments of macroencapsulation implantation devices and/or containers disclosed herein may be used with any appropriate type of macroencapsulation device as the disclosure is not limited in this fashion.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A macroencapsulation implantation device comprising:
a receptacle including an internal channel extending at least partially through and along a longitudinal axis of the receptacle;
a recess formed in a distal portion of the receptacle, wherein the recess forms a distal opening of the internal channel, and wherein the recess is sized and shaped to receive a macroencapsulation device disposed therein;
a first portion of a lock formed on the receptacle;
a handle comprising:
a second portion of the lock configured to be selectively engaged with the first portion of the lock to connect the handle to the receptacle;
a pusher configured to be inserted into the internal channel of the receptacle when the handle is connected to the receptacle, and wherein the receptacle is configured to be displaced proximally relative to the pusher to displace the macroencapsulation device out of the distal opening of the internal channel.

2. The macroencapsulation implantation device of claim 1, wherein the receptacle comprises an elongated shaft, the internal channel extending at least partially through and along a longitudinal axis of the elongated shaft.

3. The macroencapsulation implantation device of claim 2, wherein the lock is configured to be selectively moved between at least a first configuration and a second configuration, wherein in the first configuration the lock is disengaged, and wherein in the second configuration the first portion of the lock and the second portion of the lock are engaged to retain the receptacle on the handle, and wherein the lock is configured to provide at least one of a tactile click or an audible click in response to transitioning from the first configuration to the second configuration.

4. (canceled)

5. The macroencapsulation implantation device of claim 1, wherein the lock is configured to be displaced when the lock is in the second configuration to displace the receptacle relative to the pusher.

6. The macroencapsulation implantation device of claim 5, further comprising a safety configured to selectively prevent the lock from being displaced to displace the receptacle relative to the handle, wherein the safety is removeable to permit the lock to be displaced.

7. (canceled)

8. The macroencapsulation implantation device of claim 1, wherein a width of a transverse cross section of the recess in a first direction perpendicular to the longitudinal axis of the receptacle is greater than a thickness of the transverse cross section of the recess in a second direction perpendicular to the longitudinal axis and the first direction and wherein the width of the transverse cross section of the recess is less than or equal to a width of the receptacle.

9. (canceled)

10. The macroencapsulation implantation device of claim 1, wherein a width of the pusher is less than the width of the transverse cross section of the recess.

11. The macroencapsulation implantation device of claim 1, wherein a distal portion of the pusher is curved to complement a shape of a proximal portion of the macroencapsulation device, and wherein a curvature of the distal portion of the pusher in a thickness direction of the pusher is different from a curvature of the distal portion of the pusher in a width direction of the pusher.

12. (canceled)

13. The macroencapsulation implantation device of claim 1, further comprising a cover configured to be selectively retained on the distal portion of the receptacle, and wherein the cover is configured to at least partially cover the distal opening of the receptacle when positioned on the distal portion of the receptacle, wherein the cover includes a support shaft configured to extend distally away from the distal portion of the receptacle when the cover is positioned on the receptacle, the macroencapsulation implantation device further comprising one or more detents configured to selectively retain the cover on the distal portion of the receptacle.

14-15. (canceled)

16. The macroencapsulation implantation device of claim 1, further comprising the macroencapsulation device disposed in the recess.

17. The macroencapsulation device of claim 1, further comprising a plurality of length indicators disposed at intervals along a length of the device, the plurality of length indicators configured to indicate a depth of insertion during an implantation procedure.

18. A method of implanting a macroencapsulation device, the method comprising:
inserting a pusher of a handle into an internal channel of a receptacle;
connecting the handle to the receptacle with a lock;
proximally displacing the receptacle relative to the pusher and handle to displace a macroencapsulation device disposed in a recess formed in a distal portion of the receptacle out of a distal opening of the internal channel.

19. The method of claim 18, further comprising moving the lock to a first configuration to selectively disengage the lock, and moving the lock to a second configuration to lock and retain the receptacle on the handle, wherein moving the lock to the second configuration comprises providing at least one of a tactile click or an audible click.

20. (canceled)

21. The method of claim 18, wherein proximally displacing the receptacle relative to the handle and pusher comprises displacing the lock when the lock is in the second configuration, the method further comprising selectively preventing displacement of the lock in the second configuration with a safety, and removing the safety to permit the lock to be displaced in the second configuration.

22-23. (canceled)

24. The method of claim 18, further comprising selectively retaining a cover on the distal portion of the receptacle, wherein the cover is configured to at least partially cover the distal opening of the receptacle when positioned on the distal portion of the receptacle, the method further comprising removing the cover prior to proximally displacing the receptacle relative to the pusher.

25. (canceled)

26. A macroencapsulation implantation device comprising:
a receptacle including an internal channel extending at least partially through and along a longitudinal axis of the receptacle;
a recess formed in a distal portion of the receptacle, wherein the recess forms a distal opening of the internal channel, and wherein the recess is sized and shaped to receive a macroencapsulation device disposed therein;
a plurality of slots formed in the distal portion of the receptacle and extending in a direction substantially parallel to the longitudinal axis of the receptacle, wherein the plurality of slots is configured to place the recess in fluid communication with a surrounding environment.

27-33. (canceled)

34. A method of storing a macroencapsulation device, the method comprising:
transferring nutrients and waste between the macroencapsulation device and a surrounding media through a plurality of slots formed in a distal portion of a receptacle the macroencapsulation device is disposed in.

35-40. (canceled)

41. A container system for storing a macroencapsulation device, the container comprising:
a body including an internal volume and an opening;
a pair of rails positioned on opposing sides of a longitudinal axis of the internal volume and extending into the internal volume,
an opposing pair of grooves formed in and extending along at least a portion of a longitudinal length of the pair of rails, and wherein the pair of rails are configured to retain a correspondingly sized and shaped receptacle engaged with the opposing pair of grooves in a predetermined pose in the internal volume.

42-56. (canceled)

57. A method of storing a macroencapsulation device, the method comprising:
inserting a receptacle into an opposing pair of grooves formed in and extending along at least a portion of a longitudinal length of a pair of rails positioned on opposing sides of a longitudinal axis of an internal volume of a container, wherein the macroencapsulation device is disposed in a recess formed in a distal portion of the receptacle; and
supporting the receptacle in a predetermined pose within the internal volume of the container with the opposing pair of grooves.

58-64. (canceled)

65. A macroencapsulation implantation device comprising:
a receptacle including an internal channel extending at least partially through and along a longitudinal axis of the receptacle;
a recess formed in a distal portion of the receptacle, wherein the recess forms a distal opening of the internal channel, and wherein the recess is sized and shaped to receive a macroencapsulation device disposed therein;
a pusher configured to be inserted into the internal channel of the receptacle when the handle is connected to the receptacle, and wherein the receptacle is configured to be displaced proximally relative to the pusher to displace the macroencapsulation device out of the distal opening of the internal channel; and
a plurality of length indicators disposed at intervals along a length of the device, the plurality of length indicators configured to indicate a depth of insertion during an implantation procedure.

66-82. (canceled)
